SEQUENCE LISTING (32 sequences)

SEQ ID NO: 1 - RNA, 58 nt, Arabidopsis thaliana
uacaucacaaucacacaaaacuaacaaaagaucaaaagcaaguucuucacuguugaua

SEQ ID NO: 2 - RNA, 82 nt, Arabidopsis thaliana
cuucacaauccucauaaucacuuucgaaauuacauuuacgcuuucuugcaaucaaauuuuccgaucuuaaguucagaagacg

SEQ ID NO: 3 - RNA, 177 nt, Arabidopsis thaliana
gguagaaaaaccauucuuugguaacuuucgcuuacucucuccccauucucucucgaagagagccucgccggaguuuguuuuucuucuuguuucgcggccaaaaccgccuuuguguaaagcuaccuuguuauuuuccuccguuaaccaucugguauagguugcugcuggacucuuuag

SEQ ID NO: 4 - RNA, 117 nt, Arabidopsis thaliana
acacaagcauuuucaaggauaucaaaucacaaucccaagaagagcaauaacaagagaagaagaaguaguucaagaauuaaggaagagagcuucuccguuaaaguauagugagagaau

SEQ ID NO: 5 - RNA, 173 nt, Arabidopsis thaliana
ccgucgucgaugucgagaauuaucaccaucuagaaucuucccucaaccucuagaaaacaacuuucguauccuucaaaaacccuaaacccuagcuuuugcacagacgcaauuucaucgcaucauuuugcaauuuuccuuuacugaucugucaccaccucuugaauuucgaaacc

SEQ ID NO: 6 - RNA, 81 nt, Arabidopsis thaliana
aucaucacaacacaaaucaaaacaagaauaacaaaaucuuucucuuauaaauucuuauuucaagacaucaaaggagaauua

SEQ ID NO: 7 - DNA, 22 bp, Artificial
  PCR primer
tcccattgttttgtagctctga

SEQ ID NO: 8 - DNA, 26 bp, Artificial
  PCR primer
aaagaaattatagggaacaaaaggaa

SEQ ID NO: 9 - DNA, 21 bp, Artificial
  PCR primer
tgccaaggaatatctcgacaa

SEQ ID NO: 10 - DNA, 20 bp, Artificial
  PCR primer
ctgaactggctgctacatgg

SEQ ID NO: 11 - DNA, 22 bp, Artificial
  PCR primer
gagtattcgttgcatcatcacc

SEQ ID NO: 12 - DNA, 21 bp, Artificial
  PCR primer
caaagtgaacatcatctgcga

SEQ ID NO: 13 - DNA, 21 bp, Artificial
  PCR primer
tgctgcaactaagaggaaagc

SEQ ID NO: 14 - DNA, 20 bp, Artificial
  PCR primer
ccttggctggtctagcctta

SEQ ID NO: 15 - DNA, 21 bp, Artificial
  PCR primer
ttttcctccgttaaccatctg

SEQ ID NO: 16 - DNA, 21 bp, Artificial
  PCR primer
tccgagagtctgccatctaaa

SEQ ID NO: 17 - DNA, 20 bp, Artificial
  PCR primer
aacaacaagcgcaagaacaa

SEQ ID NO: 18 - DNA, 20 bp, Artificial
  PCR primer
cgatttgtgtttttgcagga

SEQ ID NO: 19 - DNA, 21 bp, Artificial
  PCR primer
agcttttccagagcctttagc

SEQ ID NO: 20 - DNA, 23 bp, Artificial
  PCR primer
cccaaatcaataccaatgacatc

SEQ ID NO: 21 - DNA, 18 bp, Artificial
  PCR primer
acggctgctaccgttgaa

SEQ ID NO: 22 - DNA, 20 bp, Artificial
  PCR primer
ggtacaaacatggcagcatc

SEQ ID NO: 23 - DNA, 20 bp, Artificial
  PCR primer
ctgatagcgcgtgacaaaaa

SEQ ID NO: 24 - DNA, 19 bp, Artificial
  PCR primer
cggttcgttggcaatactc

SEQ ID NO: 25 - DNA, 23 bp, Artificial
  PCR primer
tggcatcatacattttacaacga

SEQ ID NO: 26 - DNA, 24 bp, Artificial
  PCR primer
aggtgcttcagtgagtagtacagg

SEQ ID NO: 27 - DNA, 20 bp, Artificial
  PCR primer
gggaggtccttcttggagag

SEQ ID NO: 28 - DNA, 21 bp, Artificial
  PCR primer
gcaagatccagaaatgcttgt

SEQ ID NO: 29 - DNA, 24 bp, Artificial
  PCR primer
ggattcttttccaatgctattgtt

SEQ ID NO: 30 - DNA, 27 bp, Artificial
  PCR primer
aagaccttttactttgacaaattcagt

SEQ ID NO: 31 - DNA, 23 bp, Artificial
  PCR primer
tgagtacttcgaaatgtccgttc

SEQ ID NO: 32 - DNA, 23 bp, Artificial
  PCR primer
gtattcagcccatatcgtttcat

(19) United States
(12) Patent Application Publication
(10) Pub. No.: US 2014/0315248 A1
(43) Pub. Date: Oct. 23, 2014

(54) PROTEIN PRODUCTION METHOD USING TRANSFORMED PLANT CELLS

(71) Applicant: NATIONAL UNIVERSITY CORPORATION NARA INSTITUTE OF SCIENCE AND TECHNOLOGY, Nara (JP)

(72) Inventors: Ko Kato, Ikoma-shi (JP); Kiyotaka Ueda, Ikoma-shi (JP); Renya Okawara, Ikoma-shi (JP); Toshihiro Yamura, Ikoma-shi (JP)

(21) Appl. No.: 14/240,581

(22) PCT Filed: Aug. 29, 2012
(86) PCT No.: PCT/JP2012/071815

§ 371 Date: Feb. 24, 2014

(30) Foreign Application Priority Data
Sep. 2, 2011 (JP) 2011-191180

(51) Int. Cl. C12P 21/00

(52) U.S. Cl. CPC C12P 21/00
USPC 435/69.1; 536/23.1

(57) ABSTRACT

An object of this invention is to produce a protein by efficiently culturing plant cells while escaping mRNA translational repression in cultured plant cells under stress caused by lack of conditions essentially required for the growth (e.g., nutrient-starvation stress and hypoxic stress). This invention provides a method for escaping translational repression of a protein encoded by mRNA, the repression being induced by stress due to absence of conditions required for the growth, the method comprising the step of culturing a plant cell transformed with a recombinant DNA molecule encoding mRNA containing 5′ UTR defined in (a) or (b) below, the culturing being carried out under stress due to absence of conditions required for the growth: (a) 5′ UTR having a base sequence of SEQ ID NO: 1, 2, 3, 4, 5, or 6; or (b) 5′ UTR having the base sequence of the 5′ UTR of (a) in which one or more bases are replaced, deleted, or added, and which escapes translational repression induced by stress due to absence of conditions required for the growth.

### TECHNICAL FIELD

The present invention relates to a method for producing a protein using transformed plant cells. More specifically, the invention relates to a method for producing a protein using plant cells transformed with a recombinant gene encoding a specific 5′ untranslated region (5′ UTR).

### BACKGROUND ART

Methods for producing useful proteins using microorganisms, animal cells, or plant cells have been widely known. A target protein can be produced by introducing a gene that encodes the target protein into microorganisms, animal cells, or plant cells to obtain a transformant, and culturing this transformant.

However, when culturing microorganisms and cells, the efficiency in culturing and the efficiency in translation into protein sometimes decrease due to environmental stress. Recent research suggests a relationship between 5′ UTR and changes in translational state caused by stress. There is a report stating that an important region and sequence of 5′ UTR, which regulates translational control, were identified by studying this relationship (PTL 1). However, this report only discloses the escape from translational repression induced by stress caused by conditions not required for the growth, such as temperature stress or osmotic pressure stress (salt stress), and nowhere suggests a relationship between translational repression and stress caused by absence of conditions essentially required for the growth, such as nutrient-starvation stress.

In general, when a useful protein is produced using cultured plant cells, no environmental stress (e.g., temperature stress and salt stress) is applied, and the culture conditions are fully optimized. In particular, application of stress caused by absence of conditions essentially required for the growth (hereinafter this stress is referred to as “stress due to absence of conditions required for the growth”) generally decreases the production amount of a useful protein intended for production. One of the reasons for this is considered to be the result of a decrease in protein production efficiency due to a repression of mRNA translation. In view of this, in order to produce proteins by efficiently culturing plant cells, it is important to escape translational repression and maintain protein production efficiency.

### CITATION LIST

### Patent Literature

PTL 1: WO 2011/021666

### SUMMARY OF INVENTION

### Technical Problem

An object of the invention is to produce a protein by efficiently culturing plant cells while escaping mRNA translational repression in cultured plant cells, in particular, under stress caused by lack of conditions that are essentially required for the growth (stress due to absence of conditions required for the growth, such as nutrient-starvation stress and hypoxic stress).

### Solution to Problem

In order to achieve the above object, the present inventors conducted extensive research, and found that translational repression would not occur in mRNA containing a specific 5′ UTR sequence, even when this mRNA is put under stress due to absence of conditions required for the growth. The present inventors conducted further research, and thereby accomplished the invention.

More specifically, as described in the items below, the invention encompasses, for example, a method for escaping translational repression induced by stress due to absence of conditions required for the growth, and a method for producing a protein.

### Item 1.

A method for escaping translational repression of a protein encoded by mRNA, the repression being induced by stress due to absence of conditions required for the growth,

the method comprising the step of culturing a plant cell transformed with a recombinant DNA molecule encoding mRNA containing 5′ UTR defined in (a) or (b) below, the culturing being carried out under stress due to absence of conditions required for the growth:

(a) 5′ UTR having a base sequence of SEQ ID NO: 1, 2, 3, 4, 5, or 6; or
(b) 5′ UTR having the base sequence of the 5′ UTR of (a) in which one or more bases are replaced, deleted, or added, and which escapes translational repression induced by stress due to absence of conditions required for the growth.

### Item 2.

The method according to Item 1, wherein the stress due to absence of conditions required for the growth is at least one stress selected from the group consisting of nutrient-starvation stress and hypoxic stress.

### Item 3.

The method according to Item 1 or 2, wherein the plant cell is an Arabidopsis thaliana cultured cell or a tobacco cultured cell.

### Item 4.

A method for producing a protein by escaping translational repression of a protein encoded by mRNA, the repression being induced by stress due to absence of conditions required for the growth,

the method comprising the step of culturing a plant cell transformed with a recombinant DNA molecule encoding mRNA containing 5′ UTR defined in (a) or (b) below, the culturing being carried out under stress due to absence of conditions required for the growth:

(a) 5′ UTR having a base sequence of SEQ ID NO: 1, 2, 3, 4, 5, or 6; or
(b) 5′ UTR having the base sequence of the 5′ UTR of (a) in which one or more bases are replaced, deleted, or added, and which escapes translational repression induced by stress due to absence of conditions required for the growth.

### Item 5.

The method according to Item 4, wherein the stress due to absence of conditions required for the growth is at least one stress selected from the group consisting of nutrient-starvation stress and hypoxic stress.

### Item 6.

The method according to Item 4 or 5, wherein the plant cell is an Arabidopsis thaliana cultured cell or a tobacco cultured cell.

### Item A.

RNA containing a base sequence of SEQ ID NO: 2, 3, 5 or 6.

### Item B.

DNA encoding RNA of Item A.

### Item C.

Recombinant DNA containing a region encoding the RNA of Item A.

### Advantageous Effects of Invention

According to the invention, in producing a protein by culturing plant cells transformed by the introduction of a gene encoding the protein, the efficiency in mRNA-protein translation does not decrease even under stress due to absence of conditions required for the growth, such as nutrient-starvation stress and hypoxic stress. It is thereby possible to efficiently produce a target protein.

### BRIEF DESCRIPTION OF DRAWINGS

FIG. 1 shows the growth curve (A) of wild-type Arabidopsis thaliana T87 strain, and the results of polysome analysis (B).

(A): After subculture, the fresh weights (g/mL) of cultured cells present per mL of culture medium were measured every other day for 9 days. The averages and standard deviations of the measured weights (3 times each) are shown.

(B): Cell extracts prepared from the recovered cells were fractionated by sucrose density-gradient centrifugation (15-60%), and the absorbance profiles at 254 nm were then recorded. (The results of days 0, 1, 3, 5, and 8 are shown.) The graphs show polysome fractions and non-polysome fractions. The sedimentation direction is from right to left.

FIG. 2 shows the overview of polysome/microarray analysis. Cell extracts prepared from the cells on days 1 and 8 of culture were fractionated by sucrose density-gradient centrifugation (15-60%), and the absorbance profiles at 254 nm were then recorded to estimate the distribution of RNA. Each density gradient solution was fractionated into 8 fractions in equal amounts, and polysome RNA was extracted and purified from fraction mixtures 1 to 3 while total RNA was extracted and purified from all 8 fractions. Using each RNA as a template, cRNA fluorescently labeled with Cy3 or Cy5 was prepared and subjected to a microarray hybridization experiment. For each mRNA species, the ratio of polysome RNA to total RNA (Polysome Ratio: PR) was calculated with regard to day 1 (PR_{1d}) and day 8 (PR_{8d}). ΔPR is an index indicating changes in the PR values between day 1 and day 8 (i.e., changes in the translational state).

FIG. 3 shows the results of genome-wide analysis in terms of the state of polysome formation in the first culture period (day 1) and the late culture period (day 8). The changes in the state of polysome formation indicate changes in translational state under stress due to absence of conditions required for the growth. FIG. 3(A) shows histograms of PR values on days 1 and 8 of culture. The ordinate shows the number of genes. FIG. 3(B) shows a ΔPR histogram of 17077 types of mRNA. ΔPR is an index indicating changes in translational state. The ordinate shows the number of genes. It is shown that there is a small amount of mRNA whose translation was maintained (ΔPR>0).

FIG. 4 shows the overview of polysome/quantitative RT-PCR analysis. Cell extracts prepared from the cells on days 1 and 8 of culture were fractionated by sucrose density-gradient centrifugation (15-60%), and the absorbance profiles at 254 nm were then recorded to estimate the distribution of RNA. Each density gradient solution was fractionated into 8 fractions in equal amounts, and the mRNA was purified from each fraction and subjected to quantitative RT-PCR. The behavioral changes of the endogenous genes (At1g77120, At1g06760, At4g10710, At4g14560, At5g09590, At1g55330, At3g47610, and act2) present in the fractions were analyzed using quantitative RT-PCR. The gene act2 is an actin gene.

FIG. 5A shows the results of polysome/quantitative RT-PCR analysis in terms of endogenous genes in Arabidopsis thaliana.

(A): Cell extracts prepared from the cells on days 1 and 8 of culture were fractionated by sucrose density-gradient centrifugation (15-60%), and the absorbance profiles at 254 nm were then recorded to estimate the distribution of RNA.

FIG. 5B shows the results of polysome/quantitative RT-PCR analysis in terms of endogenous genes in Arabidopsis thaliana.

(B): RNA extracted from each fraction was subjected in equal amounts to quantitative RT-PCR analysis, and the endogenous gene mRNA present in each fraction was quantified. The ordinate shows the mRNA level in each fraction relative to that in all of the fractions.

FIG. 6 schematically illustrates binary vectors produced for use in production of transformed Arabidopsis thaliana cells.

FIG. 7 shows the results of GUS mRNA behavioral analysis in terms of cultured cells transformed with At3g47610.

(A): Cell extracts prepared from the cells on days 1 and 8 of culture were fractionated by sucrose density-gradient centrifugation (15-60%), and the absorbance profiles at 254 nm were then recorded to estimate the distribution of RNA.

(B): RNA extracted from each fraction was subjected in equal amounts to quantitative RT-PCR analysis, and GUS mRNA containing the 5′ UTR of act2, At3g47610, At1g77120, or At3g47610 present in each fraction was quantified. The ordinate shows the mRNA level in each fraction relative to that in all of the fractions.

FIG. 8 shows the results of GUS mRNA behavioral analysis in terms of cultured cells transformed with At1g77120.

(A): Cell extracts prepared from the cells on days 1 and 8 of culture were fractionated by sucrose density-gradient centrifugation (15-60%), and the absorbance profiles at 254 nm were then recorded to estimate the distribution of RNA.

(B) RNA extracted from each fraction was subjected in equal amounts to quantitative RT-PCR analysis, and GUS mRNA containing the 5′ UTR of act2, At3g47610, At1g77120, or At1g77120 present in each fraction was quantified. The ordinate shows the mRNA level in each fraction relative to that in all of the fractions.

FIG. 9 schematically illustrates binary vectors produced for use in the production of transformed Arabidopsis thaliana cells.

FIG. 10 shows the results of Fluc mRNA behavioral analysis in terms of cultured cells transformed with At3g47610.

(A): Cell extracts prepared from the cells on days 1, 4, and 9 of culture were fractionated by sucrose density-gradient centrifugation (15-60%), and the absorbance profiles at 254 nm were then recorded to estimate the distribution of RNA.

(B): RNA extracted from each fraction was subjected in equal amounts to quantitative RT-PCR analysis, and Fluc mRNA containing the 5′ UTR of act2, At3g47610, At1g77120, or At3g47610 present in each fraction was quantified. The ordinate shows the mRNA level in each fraction relative to that in all of the fractions.

FIG. 11 shows the results of Fluc mRNA behavioral analysis in terms of cultured cells transformed with At1g77120.

(A): Cell extracts prepared from the cells on days 1, 4, and 9 of culture were fractionated by sucrose density-gradient centrifugation (15-60%), and the absorbance profiles at 254 nm were then recorded to estimate the distribution of RNA.

(B): RNA extracted from each fraction was subjected in equal amounts to quantitative RT-PCR analysis, and Fluc mRNA containing the 5′ UTR of act2, At3g47610, At1g77120, or At1g77120 present in each fraction was quantified. The ordinate shows the mRNA level in each fraction relative to that in all of the fractions.

FIG. 12 shows the results of analysis regarding influence of 5′ UTR on the accumulated amount of Fluc protein. After subculture, cells cultured for 4 to 9 days were collected. Crude protein solutions were prepared from the collected cells, and the Fluc activity of the total amount of the proteins was measured. The relative Fluc activity (%) is shown based on the activity of the transformed cultured cells on day 4 taken as 100. The Fluc activity level was measured 5 times each, and the averages and standard deviations of Fluc activity level were calculated using the measured activity levels of 3 times each, with the maximum and minimum levels excluded.

FIG. 13 schematically illustrates binary vectors produced for use in production of transformed tobacco cells.

FIG. 14 shows the results of HRP C1a mRNA behavioral analysis in terms of cultured cells transformed with At3g47610.

(A): After subculture, the fresh weights (g/mL) of cultured cells present per mL of culture medium were measured every other day for 9 days. The averages and standard deviations of the measured weights (3 times each) are shown.

(B): Cell extracts prepared from the cells on days 1 and 8 of culture were fractionated by sucrose density-gradient centrifugation (15-60%), and the absorbance profiles at 254 nm were then recorded to estimate the distribution of RNA.

(C): RNA extracted from each fraction was subjected in equal amounts to quantitative RT-PCR analysis, and HRP C1a mRNA containing the 5′ UTR of T-actin or At3g47610 present in each fraction was quantified. The ordinate shows the mRNA level in each fraction relative to that in all of the fractions. The T-actin is a tobacco actin gene.

FIG. 15 shows the results of HRP C1a mRNA behavioral analysis in terms of cultured cells transformed with At1g77120.

(A): After subculture, the fresh weights (g/mL) of cultured cells present per mL of culture medium were measured every other day for 9 days. The averages and standard deviations of the measured weights (3 times each) are shown.

(B): Cell extracts prepared from the cells on days 1 and 8 of culture were fractionated by sucrose density-gradient centrifugation (15-60%), and the absorbance profiles at 254 nm were then recorded to estimate the distribution of RNA.

(C): RNA extracted from each fraction was subjected in equal amounts to quantitative RT-PCR analysis, and HRP C1 mRNA containing the 5′ UTR of T-actin or At1g77120 present in each fraction was quantified. The ordinate shows the mRNA level in each fraction relative to that in all of the fractions.

FIG. 16 shows the results of HRP C1a mRNA behavioral analysis in terms of cultured cells transformed with At1g55330.

(A): After subculture, the fresh weights (g/mL) of cultured cells present per mL of culture medium were measured every other day for 9 days. The averages and standard deviations of the measured weights (3 times each) are shown.

(B): Cell extracts prepared from the cells on days 1 and 8 of culture were fractionated by sucrose density-gradient centrifugation (15-60%), and the absorbance profiles at 254 nm were then recorded to estimate the distribution of RNA.

(C): RNA extracted from each fraction was subjected in equal amounts to quantitative RT-PCR analysis, and HRP C1 mRNA containing the 5′ UTR of T-actin or At1g55330 present in each fraction was quantified. The ordinate shows the mRNA level in each fraction relative to that in all of the fractions.

### DESCRIPTION OF EMBODIMENTS

The present invention is described in further detail below. Abbreviations of amino acids, peptides, base sequences, nucleic acids, and the like indicated in this specification are in accordance with the rules of the IUPAC and IUB, the “Guidelines for Preparing Specifications, etc., Containing Base Sequences or Amino Acid Sequences” (edited by the Japan Patent Office), and the symbols conventionally used in the relevant field. DNA stands for deoxyribonucleic acid, RNA stands for ribonucleic acid, and mRNA stands for messenger RNA.

Molecular biological manipulations such as genetic manipulation can be suitably performed by known methods. For example, such manipulation can be performed in accordance with a method described in “Molecular Cloning: A Laboratory Manual,” 3rd Edition (Cold Spring Harbor Laboratory Press), and the like, unless otherwise specified.

The method of the invention comprises the step of culturing plant cells transformed with a recombinant DNA molecule encoding mRNA containing 5′ UTR as defined in (a) or (b) below, the culturing being carried out under stress due to absence of conditions required for the growth:

(a) 5′ UTR having a base sequence of SEQ ID NO: 1, 2, 3, 4, 5, or 6; or
(b) 5′ UTR having the base sequence of the 5′ UTR of (a) in which one or more bases are replaced, and which escapes translational repression induced by stress due to absence of conditions required for the growth.

mRNA containing the 5′ UTR having the base sequence of SEQ ID NO: 1, 2, 3, 4, 5, or 6 enables the escape from translational induced by stress due to absence of conditions required for the growth.

The base sequences of SEQ ID NOs: 1 to 6 are shown below. The base sequences of these SEQ ID NOs are obtained from an Arabidopsis thaliana gene. The names of genes from which each 5′ UTR were obtained are shown in parentheses immediately after the SEQ ID NO.

(derived from At1g77120)
SEQ ID NO: 1
UACAUCACAAUCACACAAAACUAACAAAAGAUCAAAAGC
AAGUUCUUCACUGUUGAUA
(derived from At1g06760)
SEQ ID NO: 2
CUUCACAAUCCUCAUAAUCACUUUCGAAAUUACAUUUAC
GCUUUCUUGCAAUCAAAUUUUCCGAUCUUAAGUUCAGAA
GACG
(derived from At4g10710)
SEQ ID NO: 3
GGUAGAAAAACCAUUCUUUGGUAACUUUCGCUUACUCUC
UCCCCAUUCUCUCUCGAAGAGAGCCUCGCCGGAGUUUGU
UUUUCUUCUUGUUUCGCGGCCAAAACCGCCUUUGUGUAA
AGCUACCUUGUUAUUUUCCUCCGUUAACCAUCUGGUAUA
GGUUGCUGCUGGACUCUUUAG
(derived from At4g14560)
SEQ ID NO: 4
ACACAAGCAUUUUCAAGGAUAUCAAAUCACAAUCCCAAG
AAGAGCAAUAACAAGAGAAGAAGAAGUAGUUCAAGAAUU
AAGGAAGAGAGCUUCUCCGUUAAAGUAUAGUGAGAGAAU
(derived from At5g09590)
SEQ ID NO: 5
CCGUCGUCGAUGUCGAGAAUUAUCACCAUCUAGAAUCUU
CCCUCAACCUCUAGAAAACAACUUUCGUAUCCUUCAAAA
ACCCUAAACCCUAGCUUUUGCACAGACGCAAUUUCAUCG
CAUCAUUUUGCAAUUUUCCUUUACUGAUCUGUCACCACC
UCUUGAAUUUCGAAACC
(derived from At1g55330)
SEQ ID NO: 6
AUCAUCACAACACAAAUCAAAACAAGAAUAACAAAAUCU
UUCUCUUAUAAAUUCUUAUUUCAAGACAUCAAAGGAGAAUUA

In addition to the 5′ UTR having the base sequence of SEQ ID NO: 1, 2, 3, 4, 5, or 6, it is also useful in the invention to use the 5′ UTR having the base sequence of SEQ ID NO: 1, 2, 3, 4, 5, or 6 in which one or more bases are replaced, deleted, or added, and that escapes translational repression induced by stress due to absence of conditions required for the growth.

The number of bases to be replaced, deleted, or added is preferably 1 to 10 (1, 2, 3, 4, 5, 6, 7, 8, 9, or 10 bases), more preferably 1 to 5, and still more preferably 1, 2, or 3. The expression “replaced, deleted, or added” indicates that at least one base modification selected from replacement, deletion, and addition is made in one sequence. The number of bases to be replaced, deleted, or added mentioned above indicates the total number of bases replaced, deleted, and added (the number that is interpreted as the minimum total number of bases that are replaced, deleted, and added).

More specifically, the recombinant DNA molecule of the invention is a DNA molecule modified to encode mRNA containing the specific 5′ UTR sequence mentioned above. The recombinant DNA molecule of the invention may also be referred to as a DNA molecule that expresses mRNA containing the specific 5′ UTR sequence. The recombinant DNA molecule of the invention is a DNA molecule produced by artificially modifying, at the very least, the base sequence corresponding to 5′ UTR.

This recombinant DNA molecule is preferably a DNA construct, although it is not particularly limited. For example, to promote mRNA expression, a DNA construct containing a promoter sequence at the upstream of the region encoding mRNA is preferable. Specific examples include expression vectors containing a base sequence encoding mRNA. This expression vector is a DNA construct in which a DNA molecule encoding the mRNA is introduced into a cloning vector. As such a cloning vector, known cloning vectors, such as plasmid vectors (preferably, in particular, Ti plasmid), cosmid vectors, and viral vectors, may be used. Further, as the promoter sequence in the vector, known promoter sequences, such as a CaMV35S promoter derived from a cauliflower mosaic virus, may be used.

A method for artificially changing the base sequence of DNA molecule can be suitably selected for use from various known methods. For example, the base sequence of DNA molecule can be modified by the following techniques: cleaving a DNA molecule with an appropriate restriction enzyme, and subsequently ligating a new DNA fragment to the cleavage site; designing a primer pair that is not completely complementary to a target gene, and performing PCR; or using the above techniques in combination.

The recombinant DNA molecule of the invention may be a DNA molecule modified to encode mRNA containing the specific 5′ UTR sequence above, and the type of protein (including peptide) encoded by the DNA molecule is not limited. Examples of proteins (including peptides) preferably include pharmacologically active proteins. Specific examples include enzymes, transcription factors, cytokines, membrane-binding proteins, various peptide hormones (e.g., insulin, growth hormone, somatostatin), and proteins for medical use, such as vaccines and antibodies (including fragments that specifically recognize a molecule such as Fab and F(ab′)₂), and the like. Further, the recombinant DNA molecule of the invention may also be a DNA molecule prepared by ligating a reporter gene such as GFP or luciferase, a peptide tag sequence such as a His tag or FLAG-tag (registered trademark) to a DNA molecule encoding the above-described protein, or an artificially designed chimeric gene.

A known material can be used as a starting material for the recombinant DNA molecule. When a known DNA molecule is used as a starting material, the base sequence information can be obtained, for example, from a database such as the sequence database GenBank managed by the National Center for Biotechnology Information (NCBI). Based on the sequence information, a DNA molecule can be produced from various organisms by, for example, a common method such as PCR. Additionally, known genes are commercially available, for example, as a cDNA library or the like from various companies, and these genes can be purchased and used.

Introduction of the recombinant DNA molecule encoding mRNA containing the 5′ UTR above into plant cells allows expression of this mRNA in the plant cells (transformed plant cells). As long as the mRNA is expressed, the intracellular state of the introduced recombinant DNA molecule is not particularly limited. For example, it may be those that can replicate independently of chromosomal DNA (e.g., plasmid), or it may be incorporated in a chromosome.

The type of plant cells is not particularly limited. Examples of plant cells include dicotyledon-derived cells, more specifically, Arabidopsis thaliana-derived cells, tobacco-derived cells, soybean-derived cells, chrysanthemum-derived cells, lettuce-derived cells, and the like. Arabidopsis thaliana cultured cells and tobacco cultured cells are preferable.

A method for introducing the recombinant DNA molecule into plant cells is not limited and can be suitably selected from known methods. Examples of methods include electroporation method, particle gun method, methods that use Ti plasmid (for example, binary vector method and leaf disc method), and the like.

The incorporation of the vector into a host can be confirmed by known methods, such as PCR, Southern hybridization, or northern hybridization.

The transformed plant cells of the invention are transformed by introduction of the recombinant DNA molecule above. More specifically, in the transformed plant cells of the invention, the mRNA is transcribed from the recombinant DNA molecule of the invention and translated into a protein. As described earlier, the recombinant DNA molecule of the invention encodes the specific 5′ UTR sequence and is capable of escaping (reducing) translational repression induced by stress due to absence of conditions required for the growth. In this specification, to “escape translational repression” does not only mean a complete loss of translational repression, but also means a reduction in translational repression.

Culturing the transformed plant cells may be carried out by known methods whereby wild-type plant cells before transformation can be cultured. For example, when the plant cells are Arabidopsis thaliana cultured cells, a known culture method for Arabidopsis thaliana cultured cells may be used. When the plant cells are tobacco cultured cells, a known culture method for tobacco cultured cells may be used.

As described above, the stress due to absence of conditions required for the growth refers to stress caused by lack of conditions that are essentially required for the growth. In terms of plant cells, in particular, such stress is caused by lack of components required for culture and thus may be paraphrased as stress caused by lack of components required for culture. More specifically, the stress is caused by, for example, consumption of the nutrition and oxygen in culture medium. Examples of such stresses include nutrient-starvation stress and hypoxic stress. There is no limitation to the stress due to absence of conditions required for the growth, but the stress is preferably at least one stress selected from the group consisting of nutrient-starvation stress (e.g., sucrose starvation stress) and hypoxic stress.

In the invention, whether translation is repressed is determined by whether the amount of mRNA present in polysome fractions decreases. Specifically, a decrease in the amount of mRNA present in polysome fractions is interpreted as the occurrence of translational repression. When the mRNA whose translation was repressed is modified to contain the 5′ UTR of (a) or (b) above, and if the amount of this mRNA present in polysome fractions does not decrease or decreases in a lesser degree at the time point when the amount of the mRNA present in polysome fractions decreased, translational repression is considered to be escaped.

Polysome fractions are fractions containing many polysomes when such fractions are obtained by separating cell extracts (bindings of mRNA to ribosome are maintained) by sucrose density-gradient centrifugation, and fractionating the density gradient solution after centrifugation into a plurality of fractions in equal amounts. Generally, mRNA molecules with high translational efficiency bind to many ribosomes per molecule, and are thus distributed in polysome fractions having a higher sedimentation rate. In terms of the density gradient solution after ultracentrifugation, fractions with higher sucrose density contain more polysomes. More specifically, when a sucrose density gradient solution (15-60%) prepared in buffer (50 mM Tris-HCl, pH 8.5, 25 mM KCl, and 10 mM MgCl₂) is subjected to ultracentrifugation under conditions of 55,000 rpm, 50 min., and 4° C., followed by fractionation into 8 fractions in equal amounts, the first to third fractions from that with highest sucrose density (from the bottom of the centrifugal tube) are polysome fractions.

### EXAMPLES

The present invention is described below in more detail. However, the scope of the invention is not limited to the following Examples. The materials and techniques used in the experiments are first described in detail, followed by specific descriptions of the contents of the experiments and the results thereof.

### Cultured Plant Cells

The following cultured plant cells were used for the study below.

### Arabidopsis Thaliana T87

Arabidopsis thaliana cultured cells (Arabidopsis thaliana T87) (Axelos et al., 1992) that were supplied from the Plant Cell Bank in Riken Gene Bank were used. Culture was performed under the following conditions: 22° C., 18-hour light period/6-hour dark period, and a stirring rate of 120 rpm (SLK-3-FS, Nippon Medical & Chemical Instruments Co., Ltd.). 95 mL of modified LS medium (Nagata, 1992) was placed in a 300-mL Erlenmeyer flask and used. Every week, 4 mL of cells that entered stationary phase were transplanted onto 95 mL of fresh medium and subcultured. Cells obtained by subculturing (3 to 4 days) of 2 mL of cells that entered stationary phase were used as cultured cells to produce stably transformed cultured cells, which are described later.

### Tobacco BY-2

Tobacco cultured cells (Nicotiana tabacum L. cv. Bright Yellow 2) was cultured under the following conditions: 27° C., 24-hour dark period, and a stirring rate of 130 rpm (BR-3000, Taitec, Saitama, Japan). 95 mL of modified LS medium was placed into a 300-mL Erlenmeyer flask and used. Every week, 4 mL of cells that entered stationary phase were transplanted onto 95 mL of fresh medium and subcultured. The solid medium used for subculturing callus was prepared in a manner similar to that used in liquid culture, except that 0.3% of gellan gum was added before sterilization. Callus was subcultured in a Petri dish (90 mm in diameter) at 25° C. in darkness (MIR-553, Sanyo Medicasystems, Osaka).

In collecting cultured cells, the medium was removed by suction filtration, and the cells were frozen in liquid nitrogen and stored at −80° C. In collecting tobacco BY2, after suction filtration, the cells were washed with 100 mL of fresh modified LS medium, suction filtration was performed again, and the cells were frozen in liquid nitrogen and stored at −80° C.

### Polysome Analysis Using Sucrose Density Gradient Centrifugation

Polysome fractionation using sucrose density gradient centrifugation was performed according to the method of Davies et al. (Davies, E., and Abe, S. 1995, Methods Cell Biol. 50, 209-222). About 300 mg of normally cultured cells or cells cultured under stress were finely ground with a pestle and a mortar in liquid nitrogen. Thereafter, 1.5 mL of buffer U (200 mM Tris-HCl, pH 8.5, 50 mM KCl, 25 mM MgCl₂, 2 mM EGTA, 100 μg/mL heparin, 2% polyoxyethylene 10-tridecyl ether (PTE), and 1% sodium deoxycholate (DOC)) was added to the ground particles, and gently suspended. After cell debris was removed by centrifugation (15,000×g, 10 min., 4° C.), the supernatant was layered on 4.6 mL of sucrose density gradient solution (15-60%) prepared in buffer B (50 mM Tris-HCl, pH 8.5, 25 mM KCl, and 10 mM MgCl₂), followed by ultracentrifugation (SW 55 Ti rotor, 55,000 rpm, 50 min., 4° C., brake-off) (Beckman Coulter). A micropipette (40 μL calibrated pipette; Drummond) connected to a peristaltic pump (Minipuls 3; Gilson) was inserted into the sucrose density gradient from the above. The sucrose density gradient solution was then aspirated from the bottom at a rate of about 1 mL/min. At the same time, the absorbance at 254 nm was recorded using a Monitor UV-1 (GE Healthcare).

### Extraction of RNA for Microarray Analysis

The sucrose density gradient solution after ultracentrifugation was fractionated into 8 fractions in equal amounts, and polysome RNA and total RNA were respectively extracted from a polysome fraction obtained by mixing fractions 1 to 3 (the fraction at the bottom is fraction 1) and from a total fraction obtained by mixing fractions 1 to 8. Each of the fractions was collected into tubes to which 8M guanidine hydrochloride had been added beforehand to obtain a final concentration of 5.5 M. At this time, spike mix A included in a Two-Color RNA Spike-In Kit (Agilent Technologies) was added to the polysome fraction, while spike mix B was added to the total fraction. In each spike mix, ten types of transcripts containing a poly-A sequence synthesized in vitro are mixed with a 200-fold dynamic range at a known quantity ratio. The spots corresponding to these transcripts are present in the Agilent oligoarray (Arabidopsis 3 oligo microarray 44K; Agilent Technologies) used in this study. An RNA spike-in was added at the same time the sucrose density gradient solution after centrifugation was collected, and was thus to be subjected to subsequent RNA purification, labeling, and hybridization (described below). This makes it possible to estimate the actual RNA ratio (polysome RNA vs. total RNA) in the sucrose density gradient by performing correction based on the signals of spots corresponding to the RNA spike-in (Melamed and Arava, 2007). 100% ethanol was added in an amount equivalent to the mixture of sucrose solution and guanidine hydrochloride, followed by cooling overnight at −20° C. and then centrifugation (20,000×g, 45 min., 4° C.). The thus-obtained pellets were washed once with 85% ethanol, and dissolved in buffer RLT included in an RNeasy kit (Qiagen). The subsequent RNA purification was performed using the RNeasy kit (Qiagen) following the accompanying protocol. Thereafter, purification by means of LiCl sedimentation and ethanol sedimentation was further performed. The quality of RNA was tested by on-chip electrophoresis using an Agilent Bioanalyzer 2100 (Agilent Technologies).

RNA Purification from Sucrose Density Gradient Solution after Centrifugation

About 650 μL each of sucrose density gradient solution was collected into 8 tubes to which 5 ng of in vitro-synthesized Renilla luciferase (r-luc) mRNA having a cap structure and containing a poly-A sequence had been added, and to which 8 M guanidine hydrochloride had been added in advance to obtain a final concentration of 5.1 M. The synthesized r-luc mRNA was used for correction in calculating the abundance of the target mRNA in each fraction by using a quantitative RT-PCR method. 100% ethanol was added to each tube in an amount equivalent to the mixture, followed by cooling overnight at −20° C. and then centrifugation (12,000×g, 45 min., 4° C.). The obtained pellets were washed once with 85% ethanol and dried. In subsequent RNA purification, an RNeasy Mini Kit (Qiagen) was used following the accompanying protocol (DNaseI treatment was performed as an option). RNA in all of the fractions was dissolved in 30 μL of RNase-free water. The quality of purified RNA was tested by 1.5% denaturing gel electrophoresis.

### Quantitative RT-PCR

RNA solutions were obtained by purification of 8 fractions fractionated in equal amounts from the sucrose density gradient solution after ultracentrifugation, and used in equal amounts to perform reverse transcription reaction. In reverse transcription reaction, a Transcription First Strand cDNA Synthesis Kit (Roche) was used according to the accompanying protocol. The volume of the reaction system was 13 μL (oligo dT primer was used). 2 μL of 1:40 dilution of reverse-transcription reaction solution was used as a template, and PCR reaction was performed in 10 μL of reaction system using a gene-specific primer set and a LightCycler 480 SYBR Green I Master (Roche Applied Science). The sequences of the primer set used in the actual study are shown below.

Sequences of primer set used in quantitative
RT-PCR (5′ to 3′)
Act2 (At3g18780)
TCCCATTGTTTTGTAGCTCTGA
AAAGAAATTATAGGGAACAAAAGGAA
At3g47610
TGCCAAGGAATATCTCGACAA
CTGAACTGGCTGCTACATGG
At1g77120
GAGTATTCGTTGCATCATCACC
CAAAGTGAACATCATCTGCGA
At1g06760
TGCTGCAACTAAGAGGAAAGC
CCTTGGCTGGTCTAGCCTTA
At4g10710
TTTTCCTCCGTTAACCATCTG
TCCGAGAGTCTGCCATCTAAA
At4g14560
AACAACAAGCGCAAGAACAA
CGATTTGTGTTTTTGCAGGA
At5g09590
AGCTTTTCCAGAGCCTTTAGC
CCCAAATCAATACCAATGACATC
At1g55330
ACGGCTGCTACCGTTGAA
GGTACAAACATGGCAGCATC
GUS
CTGATAGCGCGTGACAAAAA
CGGTTCGTTGGCAATACTC
Fluc
TGAGTACTTCGAAATGTCCGTTC
GTATTCAGCCCATATCGTTTCAT
T-actin
TGGCATCATACATTTTACAACGA
AGGTGCTTCAGTGAGTAGTACAGG
HRP Cla
GGGAGGTCCTTCTTGGAGAG
GCAAGATCCAGAAATGCTTGT
Rluc
GGATTCTTTTCCAATGCTATTGTT
AAGACCTTTTACTTTGACAAATTCAGT

Primers were designed using the Universal ProbeLibrary Assay Design Center/ProbeFinder (Roche Applied Science). The fluorescence intensity of SYBR Green I was measured with time using a LightCycler 480 System (Roche Applied Science). Data analysis was performed using the second derivative maximum method of the LightCycler Data Analysis Software (Roche Applied Science). In order to correct the difference in RNA recovery efficiency and RT-PCR reaction efficiency among the fractions, the results of r-luc mRNA for correction, which was added when the sucrose density gradient solution was collected, were used to correct the results of the target gene in each fraction. Melting curve analysis or agarose gel electrophoresis was performed to confirm that the PCR product was a single PCR product. Based on the fact that no signals were detected in the PCR reaction in which the RNA solution not subjected to reverse transcription reaction was used as a template, it was confirmed that the signals were not from genome.

### Microarray Hybridization

The polysome RNA and total RNA from the same sucrose density gradient were used to prepare complementary RNA (cRNA) fluorescently labeled with cyanine 3 (Cy3) and complementary RNA (cRNA) fluorescently labeled with cyanine 5 (Cy5), respectively. The complementary RNAs (cRNAs) were then subjected to a competitive hybridization experiment using the Agilent oligoarray (Arabidopsis 3 oligo microarray 44 K; Agilent Technologies). In the Arabidopsis 3 oligo microarray, 44000 spots of 60-mer oligo DNA selected from the base sequences of the transcripts from Arabidopsis thaliana, the above-mentioned RNA spike-in, and the like are printed. RNA was amplified and fluorescently labeled using a Low RNA Input Fluorescent Linear Amplification Kit (Agilent Technologies). First, 500 ng of polysome RNA and total RNA were used as templates, and a reverse transcription reaction was performed with an oligo dT primer and MMLV-RT containing a T7 promoter sequence as a linker sequence. The synthesized cDNA was used as a template in the T7 RNA polymerase in vitro transcription reaction to synthesize cRNA into which CPT labeled with Cy3 (polysome RNA) or Cy5 (total RNA) was incorporated. The synthesized cRNA was purified using an RNeasy kit (Qiagen). 750 ng each of cRNAs from polysome RNA and total RNA was mixed and subjected to a hybridization reaction at 65° C. for 17 hours. After the slide was washed, scanning was performed using an Agilent Technologies Microarray Scanner (Agilent Technologies), and signals of Cy3 and Cy5 were detected.

### Microarray Data Analysis

Data was extracted from scanning images and normalized using feature extraction software (Agilent Technologies). Based on flags provided in accordance with the standard settings of feature extraction software, the following spots in terms of either Cy3 or Cy5 were disregarded from the subsequent analyses: spots in which signal values are saturated (glsSaturated, rlsSaturated); spots in which signals are not uniform (glsFeatNonUnifOL, rlsFeatNonUnifOL); spots in which gene corresponding to multiple spots is outlier (glsFeatPopnOL, rlsFeatPopnOL); and spots in which signals and background are neither positive nor significant (glsPosAndSignif, rlsPosAndSignif) (glsWellAboveBG, rlsWellAboveBG). Normalization was performed based on spots corresponding to the RNA spike-in, or using the Linear & LOWESS (Locally Weighted Linear Regression) method, which is a standard normalization method in feature extraction software (Agilent technologies). The spots remaining as an analytical object were analyzed. The abbreviations used in the analysis represent the following.

Poly_{1d}: Cy3 signal value in microarray data from polysome fraction of the cells on day 1 after subculture;

Total_{1d}: Cy5 signal value in microarray data from all fractions of the cells on day 1 after subculture;

Poly_{8d}: Cy3 signal value in microarray data from the polysome fraction of the cells on day 8 after subculture; and

Total_{8d}: Cy5 signal value in microarray data from all fractions of the cells on day 8 after subculture;

As an index for evaluating the translational state, the polysome ratio (ratio of the polysome RNA to the total RNA) was determined in terms of each spot.

### Polysome Ratio (Cells on Day 1)

PR_{1d}=Poly_{1d}/Total_{1d}

### Polysome Ratio (Cells on Day 8)

PR_{8d}=Poly_{8d}/Total_{8d}

In order to evaluate changes with time in the translational state from day 1 after subculture to day 8 after subculture, an index (ΔPR) was determined in terms of each spot.

ΔPR=PR_{8d}−PR_{1d}

A gene name or systematic name (e.g., AGI code (the Arabidopsis Genome Initiative gene code)) is given for each spot on the Arabidopsis 3 oligo microarray. Basically, one gene name (systematic name) corresponds to one spot; however, one gene name (systematic name) sometimes corresponds to several numbers of spots. Among gene names (systematic names) in which both microarray data from the cells on day 1 after subculture and microarray data from the cells on day 8 after subculture satisfy the above-mentioned data processing criteria, those having an AGI code were used to calculate the polysome ratio. The calculation above was performed using Microsoft Excel.

### Fluc Activity Measurement

The cultured cells on each day of culture were precipitated by centrifugation (9, 100×g, 1 min., 22° C.). After the medium was removed with an aspirator, the cells were frozen in liquid nitrogen and stored at −80° C. 300 μL of passive lysis buffer (Promega, USA) was added to the collected cells, and the cells were disrupted with a Handy Sonic (Tomy Seiko Co., Ltd., Tokyo). The disrupted cells were re-centrifuged (20,400×g, 5 min., 4° C.), and 200 μL of the supernatant was collected. Subsequent procedures were performed basically following the protocol of a Dual-Luciferase Reporter Assay System (Promega). The supernatant was diluted 10000-fold, and 20 μL of diluted solution was mixed with 100 μL of a substrate solution and reacted. Thereafter, a luminometer (Lumat LB 9501, Berthold, Germany) was used following the accompanying protocol. The Fluc activity was determined as a relative light unit (RLU)/mg protein. The quantitation of total protein was performed by the Bradford method (Bradford, 1976). Specifically, 10 μL of diluted protein solution was added to 990 μL of protein quantification reagent, and measurements were performed using a SpectraFluor (Tecan, Switzerland). The protein concentration was determined from a standard curve created using BSA at a known concentration.

The term Fluc refers to firefly luciferase.

### Time-Dependent Changes in Translational State of Protein of Cultured Plant Cell Endogenous Gene

Polysome analysis was performed using a wild-type Arabidopsis thaliana T87 strain on each day of culture. In polysome analysis, the mRNA present in cell extracts can be fractionated by sucrose density-gradient centrifugation according to the number of bound ribosomes. For this reason, polysome analysis is widely used as a technique of analyzing translational state within the cells. This technique was used to examine changes in translational state over days of culture.

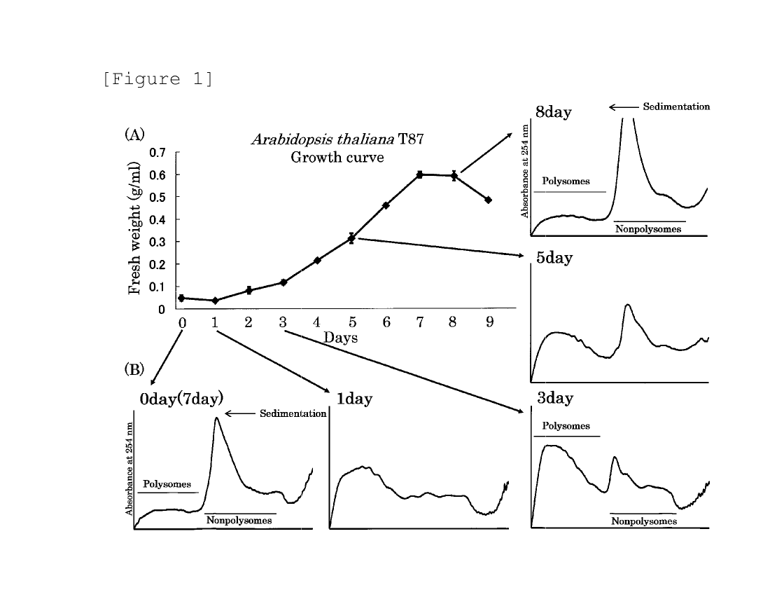

The growth curve (FIG. 1A) was created by measuring the fresh weights of the cells. The cultured cells were collected every 24 hours from day 0 (immediately after subculture) to day 9, and the cell extracts prepared from each of the cells were fractionated by sucrose density-gradient centrifugation (15-60%). On day 1 (first culture period), the majority of fractions were polysome fractions with almost no non-polysome fractions, indicating that translation was actively performed. However, with reference to the absorbance profile at 254 nm, which is an index of the RNA amount (FIG. 1B), a decrease in polysome fractions in which translation was actively performed and an increase in non-polysome fractions in which translation was inactively performed were confirmed with time during the culture. In particular, the majority of fractions obtained from the cells on day 8 (late culture period) were non-polysome fractions with lesser polysome fractions. This confirms that consumption of oxygen and nutrition caused hypoxia and nutrient-starvation stresses (stress due to absence of conditions required for the growth), resulting in significant repression in translation.

### Polysome/Microarray Analysis

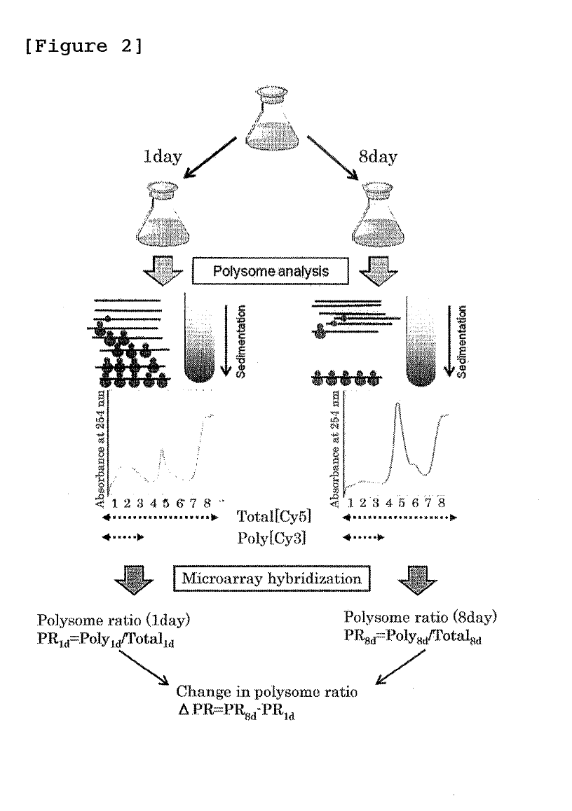

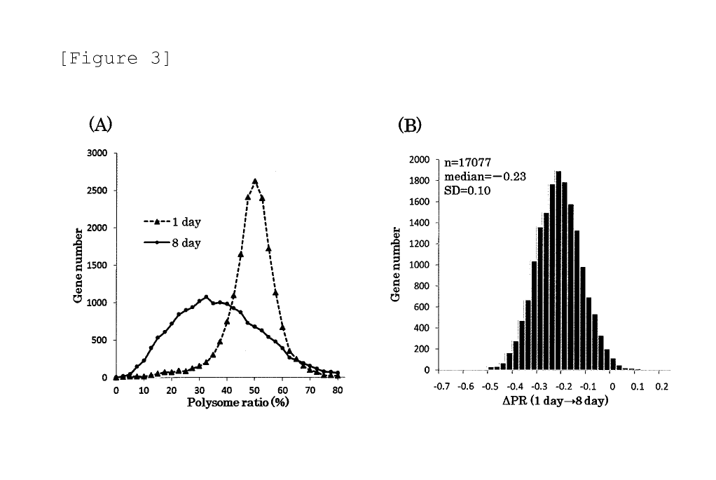

The cells on days 1 and 8 of culture were used, and mRNA was fractionated by sucrose density-gradient centrifugation according to the number of bound ribosomes. DNA microarray analysis was performed using the mRNA prepared from each fraction, and the translational state of the individual gene under stress due to absence of conditions required for the growth was genome-widely analyzed. FIG. 2 shows the overview of polysome/microarray analysis.

The polysome ratios (PR_{1d} and PR_{8d}) were calculated as indices indicating the translational state (state of polysome formation) of each mRNA within the cells. Histograms of PR values on days 1 and 8 of culture were created (FIG. 3A), and changes in translational state under stress due to absence of conditions required for the growth were examined. In terms of the cells on day 1 of culture, the majority of mRNA species had a PR_{1d} of 40 to 60%. In contrast, the PR_{8d} histogram regarding the cells on day 8 put under stress due to absence of conditions required for the growth shows a wide spread, and the majority of mRNA species had a decreased PR value. This result indicates that the proportion of each mRNA present in polysome fractions decreased because of the stress due to absence of conditions required for the growth. This agrees with the overall trend shown in the absorbance profiles at 254 nm (FIG. 1B). Further, changes in the PR values of each mRNA species from day 1 to day 8 of culture with time were calculated as ΔPR values. According to the results (FIG. 3B), the majority of mRNA species showed negative values (median −0.23), which clarifies that polysome formation was inhibited under stress due to absence of conditions required for the growth. It is also clarified that although translation was repressed in all of the cells, in a small amount of mRNAs, polysome formation was not inhibited, i.e., translation was maintained (FIG. 3B).

Endogenous mRNA Behavioral Analysis by Polysome/Quantitative RT-PCR Analysis

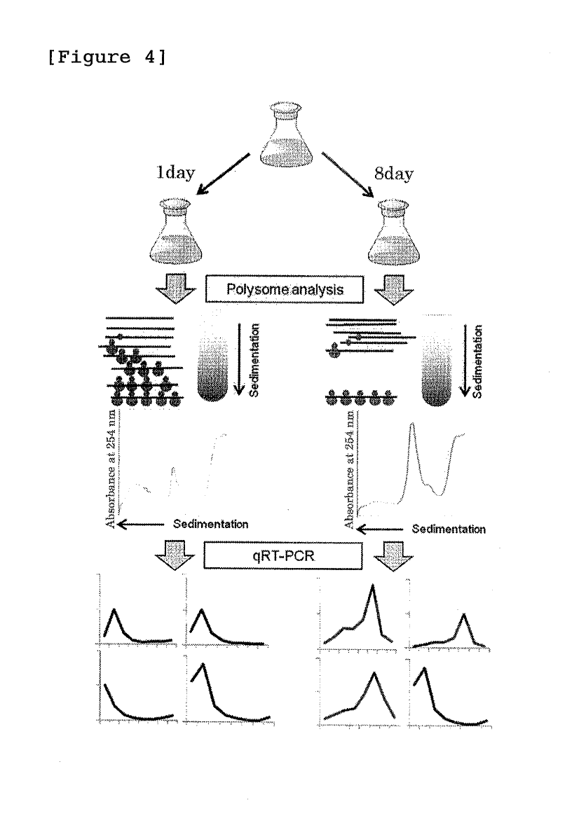

At1g77120, At1g06760, At4g10710, At4g14560, At5g09590, and At1g55330 were selected from the genes whose translation was suggested by the microarray analysis to be maintained under stress due to absence of conditions required for the growth, and subjected to behavioral analysis by using polysome/quantitative RT-PCR analysis. The genes act2 and At3g47610 whose translation was suggested to be repressed under stress due to absence of conditions required for the growth were also analyzed in a similar manner. FIG. 4 shows the overview of the polysome/quantitative RT-PCR analysis.

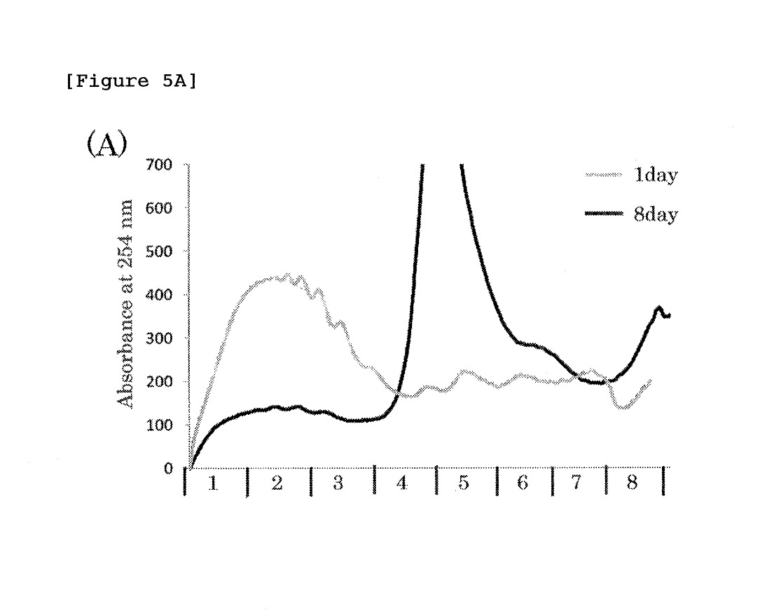

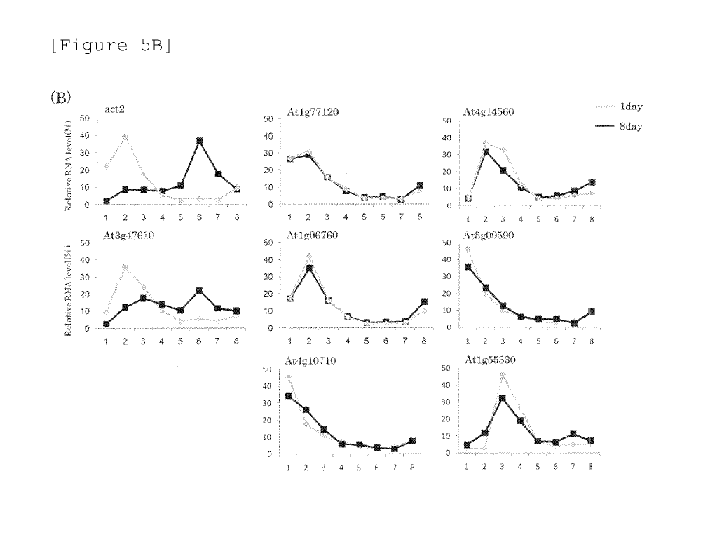

The cell extracts obtained on day 1 of culture (first culture period) and on day 8 of culture (late culture period) were fractionated by sucrose density-gradient centrifugation (15-60%). Compared to the polysome profile of the cells on day 1 of culture, a decrease in polysome fractions in which translation is actively performed and an increase in non-polysome fractions in which translation is inactively performed were confirmed on day 8 of culture, based on the absorbance profile at 254 nm, which is an index of the RNA amount (FIG. 5A). Further, the mRNAs of the above six types of genes whose translation was not repressed according to the microarray results were analyzed by quantitative RT-PCR analysis in terms of the distribution of the fractions of the sucrose density gradient solution. The results confirmed that the majority of mRNAs remained in polysome fractions 1 to 3 even on day 8 of culture, indicating that translation was maintained without being repressed (FIG. 5B). In contrast, in terms of mRNAs of housekeeping genes Actin2 (act2) and At3g47610, the majority of mRNAs were present on day 1 of culture in polysome fractions 1 to 3, indicating that translation was actively performed; however, the majority of them shifted to non-polysome fractions 4 to 7 on day 8 of culture, indicating the occurrence of translational repression (FIG. 5B).

GUS mRNA Behavioral Analysis by Polysome/Quantitative RT-PCR Analysis

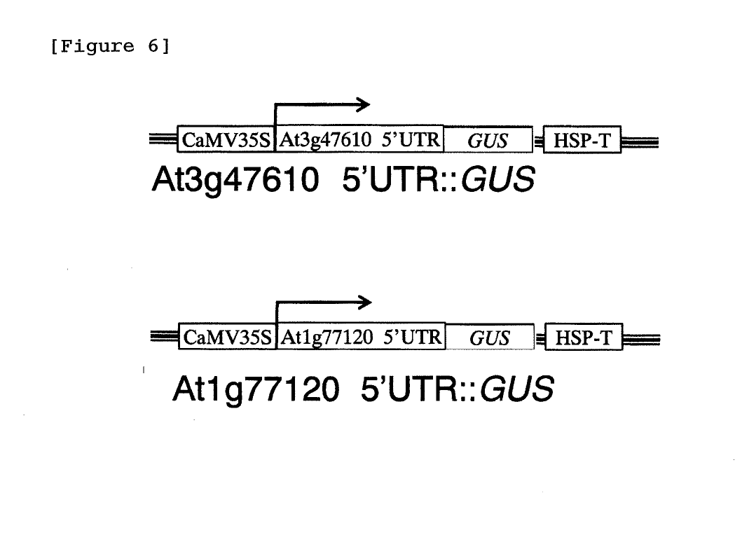

The 5′ UTRs of At1g77120, whose translation was not repressed under stress due to absence of conditions required for the growth according to the results of the above analysis, and At3g47610, whose translation was repressed under the same stress according to the results of the above analysis, were used. Specifically, the 5′ UTRs of these genes were connected to respective reporter GUS genes to produce binary vectors (“At1g77120 5′ UTR::GUS” and “At3g47610 5′ UTR::GUS”) in accordance with the method disclosed in WO 2011/021666. Arabidopsis thaliana T87 cultured cells were then introduced thereinto to produce stably transformed cultured cells. These transformed plant cells were used to perform polysome/quantitative RT-PCR analysis to thereby analyze behavioral change of the GUS mRNAs containing different 5′ UTRs and the endogenous mRNAs under stress due to absence of conditions required for the growth. The analysis was performed in a manner similar to the above. FIG. 6 schematically illustrates binary vectors produced. The HSP-T in FIG. 6 represents a HSP terminator.

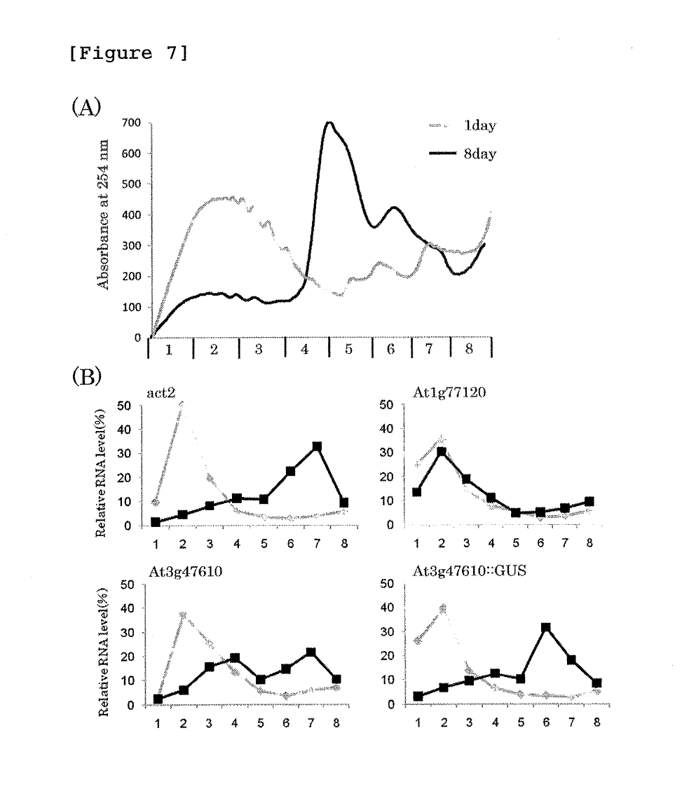

Cultured cells transformed with “At3g47610 5′ UTR::GUS” (At3g47610 5′ UTR::GUS transformed cultured cells) were subjected to polysome analysis. The results confirmed a decrease in polysome fractions and an increase in non-polysome fractions on day 8 of culture (FIG. 7A). The mRNAs were individually analyzed by quantitative RT-PCR; as a result, the mRNAs of act2 and At1g77120 showed a behavior similar to that of wild strains (FIG. 5B). The majority of the mRNA of gene At3g47610 whose translation was repressed in the late culture period was shifted to non-polysome fractions on day 8 of culture, i.e., the translation was repressed (FIG. 7B), as is the case with wild strains (FIG. 5B). The translation was also confirmed to be repressed in the GUS mRNA containing the 5′ UTR of At3g47610 (FIG. 7B).

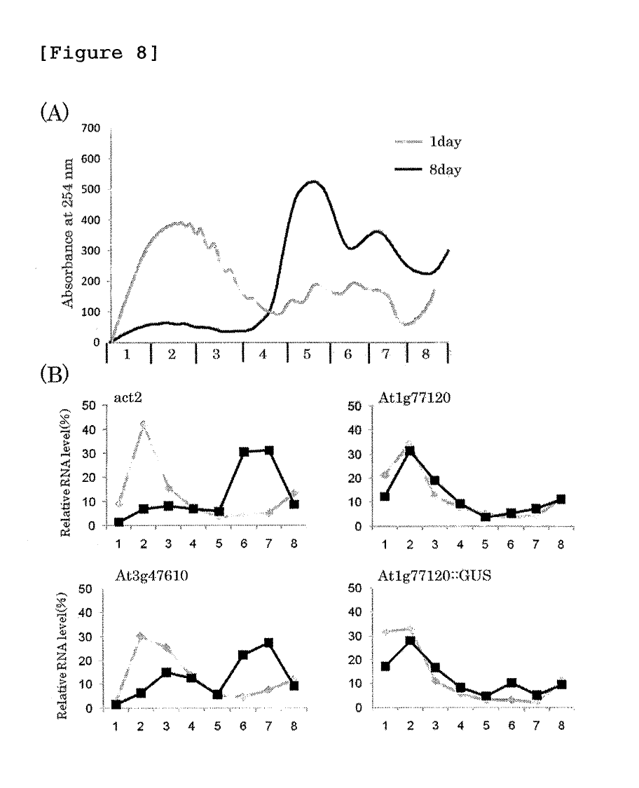

Further, cultured cells transformed with “At1g77120 5′ UTR::GUS” (At1g77120 5′ UTR::GUS transformed cultured cell) were subjected to polysome analysis. The results confirmed a decrease in polysome fractions and an increase in non-polysome fractions on day 8 of culture (FIG. 8A). In quantitative RT-PCR, the mRNAs of act2 and At3g47610 showed a behavior (FIG. 8B) similar to that of wild strains (FIG. 5B). The majority of the mRNA of gene At1g77120 whose translation was not repressed in the late culture period (FIG. 5B) and the GUS mRNA containing the 5′ UTR of At1g77120 remained in the polysome fractions on day 8 of culture, indicating that the translation was not repressed (FIG. 8B).

Fluc mRNA Behavioral Analysis by Polysome/Quantitative RT-PCR Analysis

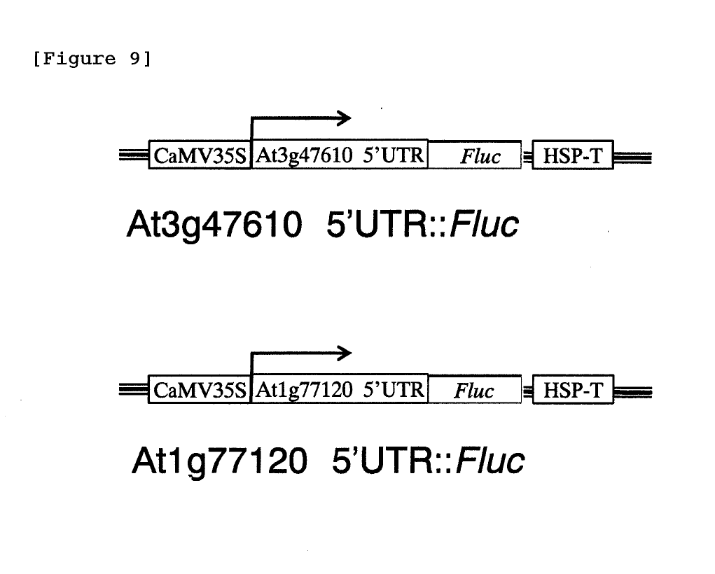

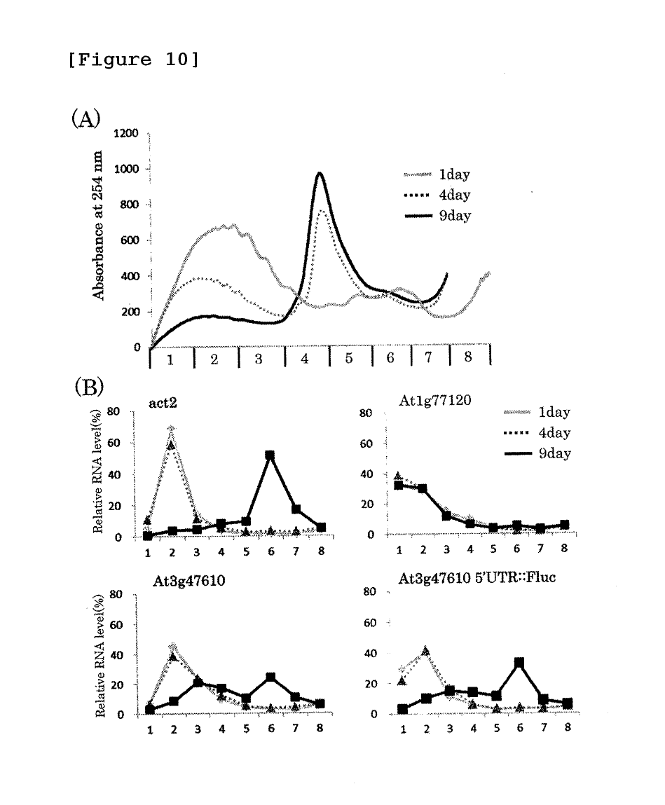

It was clarified that the GUS mRNA containing the 5′ UTR of the gene whose translation was maintained or repressed in the late culture period behaves in the late culture period in a manner similar to that of the endogenous genes. Next, this expression system was examined to confirm whether it would also function with respect to Fluc gene, which is different from GUS gene. The 5′ UTRs of At1g77120 whose translation was not repressed under stress due to absence of conditions required for the growth according to the results of the above analysis and At3g47610 whose translation was repressed under the same stress according to the results of the above analysis were used. Specifically, the 5′ UTRs of these genes were connected to respective reporter Fluc genes to produce binary vectors (“At1g77120 5′ UTR::Fluc” and “At3g47610 5′ UTR::Fluc”) in accordance with the method disclosed in WO 2011/021666. Arabidopsis thaliana T87 cultured cells were then introduced thereinto to produce stably transformed cultured cells. These transformed plant cells were used to perform polysome/quantitative RT-PCR analysis to thereby analyze behavioral change of Fluc mRNAs containing different 5′ UTRs and behavioral change of the endogenous mRNAs under stress due to absence of conditions required for the growth. The analysis was performed in a manner similar to the above. In this experiment, the cells on day 1 of culture (first culture period), day 4 of culture (middle culture period), and day 9 of culture (late culture period) were used. FIG. 9 schematically illustrates the binary vectors produced. The HSP-T in FIG. 9 represents a HSP terminator.

As a result of polysome analysis of the cultured cells transformed with “At3g47610 5′ UTR::Fluc” (At3g47610 5′ UTR::Fluc transformed cultured cells), a decrease in polysome fractions and an increase in non-polysome fractions were confirmed due to time progress from days 4 to 9 of culture (FIG. 10A). Quantitative RT-PCR confirmed that the mRNA of At1g77120, whose translation was maintained, remained in polysome fractions on both days 4 and 9 of culture. In contrast, the mRNAs of act2 and At3g47610, both of whose translation were repressed, remained in polysome fractions on day 4 of culture, but shifted to non-polysome fractions on day 9 (FIG. 10B). Further, the Fluc mRNA containing the 5′ UTR of At3g47610, whose translation was repressed in the late culture period, remained in polysome fractions on day 4 of culture, but shifted to non-polysome fractions on day 9; this indicates that the translation was repressed (FIG. 10B), as is the case with the endogenous At3g47610.

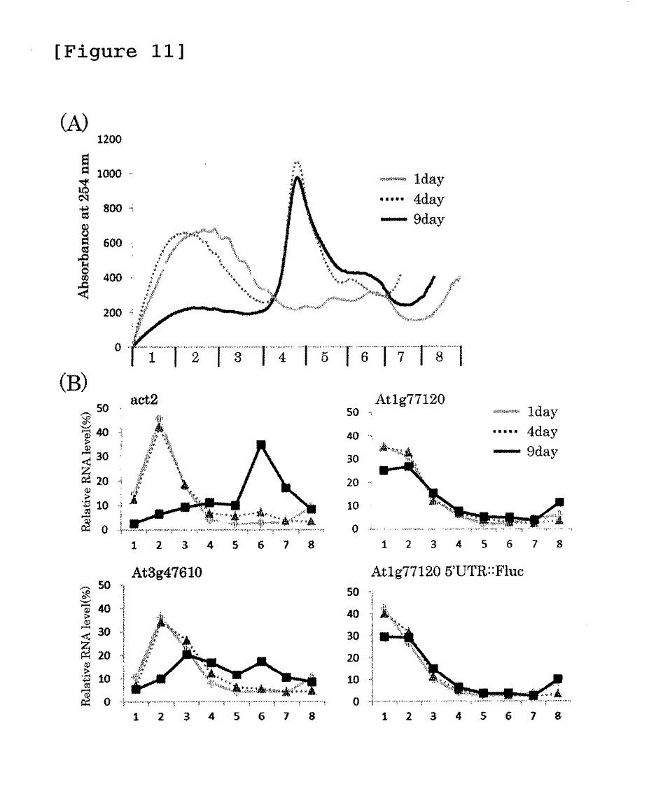

Further, as a result of polysome analysis of the cultured cells transformed with “At1g77120 5′ UTR::Fluc” (At1g77120 5′ UTR::Fluc transformed cultured cells), a decrease in polysome fractions and an increase in non-polysome fractions were confirmed on day 9 of culture (FIG. 11A). Compared to day 1, the polysome fractions moderately decreased on day 4 of culture; however, in view of the increase in non-polysome fractions, translational repression was assumed to have occurred to the same extent as the case with the At3g47610 5′ UTR::Fluc transformed cultured cells of FIG. 10, in consideration of the ratio of polysome fractions to non-polysome fractions. In quantitative RT-PCR, Fluc mRNA containing the 5′ UTR of At1g77120 whose translation was maintained in the late culture period remained in the polysome fractions on day 9 of culture, indicating that the translation was maintained (FIG. 11B), as is the case with the endogenous At1g77120.

### Fluc Protein Accumulated Amount on Each Day of Culture

Changes in the accumulated amount of Fluc protein, which is a translation product, were analyzed to examine whether the translation was actually performed in the late culture period in terms of the Fluc genes containing the 5′ UTRs that maintain translation.

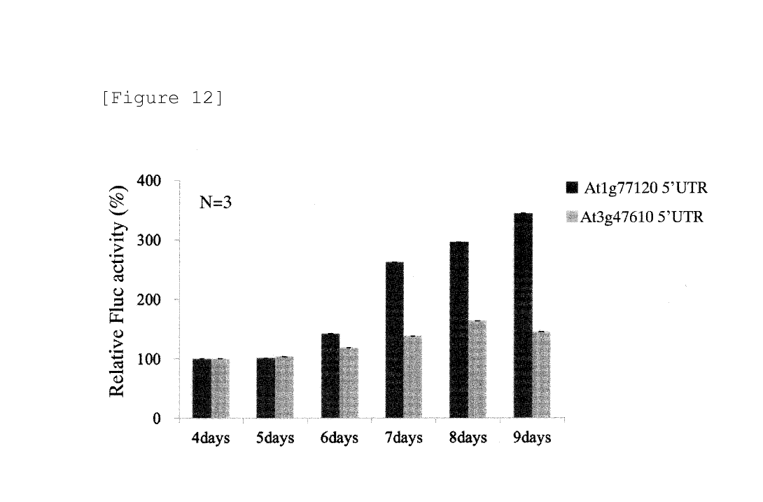

The Fluc activity of the cells on days 4 to 9 of culture was measured in relation to At1g77120 5′ UTR::Fluc whose translation was maintained in the late culture period and At3g47610 5′ UTR::Fluc transformed cultured cells whose translation was repressed in the late culture period. The results regarding At3g47610 5′ UTR::Fluc transformed cultured cells whose translation was repressed showed a gradual increase in the accumulated amount over days 4 to 8; however, the amount decreased on day 9, compared to the results of day 8 (FIG. 12). In contrast, regarding At1g77120 5′ UTR::Fluc transformed cultured cells whose translation was maintained, the accumulated amount of Fluc protein continued to greatly increase until day 9, and the accumulated amount on day 9 was 3 times the amount of day 4 (FIG. 12).

The above results clarified that when the 5′ UTR of a gene whose translation is repressed under stress due to absence of conditions required for the growth is added to a transgene, the translation is repressed under stress due to absence of conditions required for the growth, as is the case with the endogenous gene. The above results also clarified that when the 5′ UTR of a gene whose translation is not repressed under stress due to absence of conditions required for the growth is added to a transgene, the translation is actively performed without being repressed under stress due to absence of conditions required for the growth, as is the case with the endogenous gene.

HRP C1a mRNA Behavioral Analysis by Polysome/Quantitative RT-PCR Analysis

In the above study, a reporter gene containing the 5′ UTR of Arabidopsis thaliana was introduced into Arabidopsis thaliana cultured cells, and the ability of the 5′ UTR to maintain translation was evaluated by polysome/quantitative RT-PCR analysis. The results clarified that the use of the 5′ UTR of a gene whose translation is not repressed in the late culture period achieves efficient translation of the transgene.

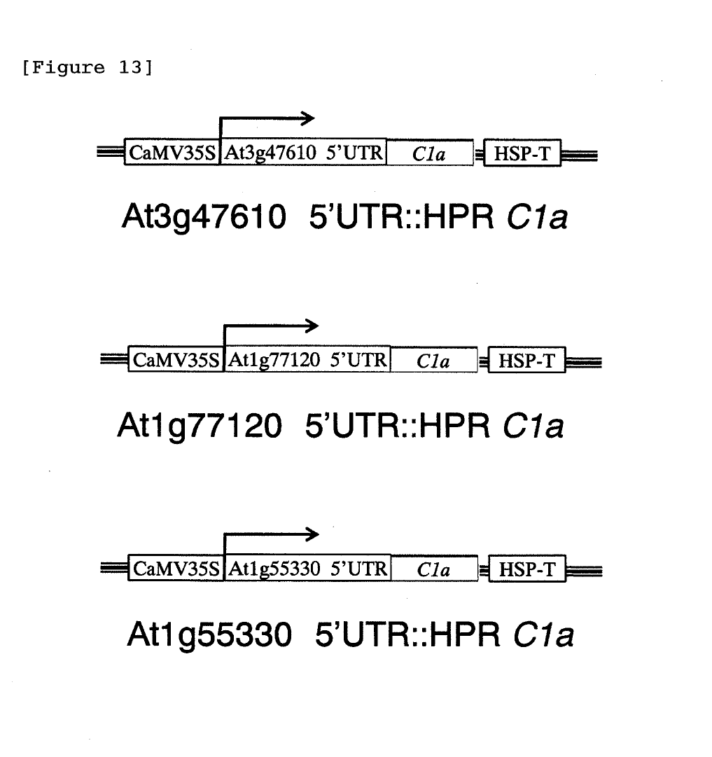

In view of the above, other cultured plant cells, i.e., tobacco BY-2 cultured cells, were also studied as to whether the same results would be attained. The 5′ UTRs of At1g77120 and At1g55330, both of whose translation was not repressed under stress due to absence of conditions required for the growth, and the 5′ UTR of At3g47610, whose translation was repressed under the same stress, were connected to respective model gene, i.e., horseradish peroxidase C1a (HRP C1a), to produce binary vectors (“At1g77120 5′ UTR::HRP C1a,” “At1g55330 5′ UTR::HRP C1a,” and “At3g47610 5′ UTR::HRP C1a”), in accordance with the method disclosed in WO 2011/021666. Tobacco BY-2 cultured cells were introduced into the resulting vectors to produce stably transformed cultured cells, and polysome/quantitative RT-PCR analysis was performed in a manner similar to the above. FIG. 13 schematically illustrates the binary vectors produced. The HSP-T in FIG. 13 represents an HSP terminator.

Tobacco Transformed Cultured Cells (At3g47610 5′ UTR::HRP C1a)

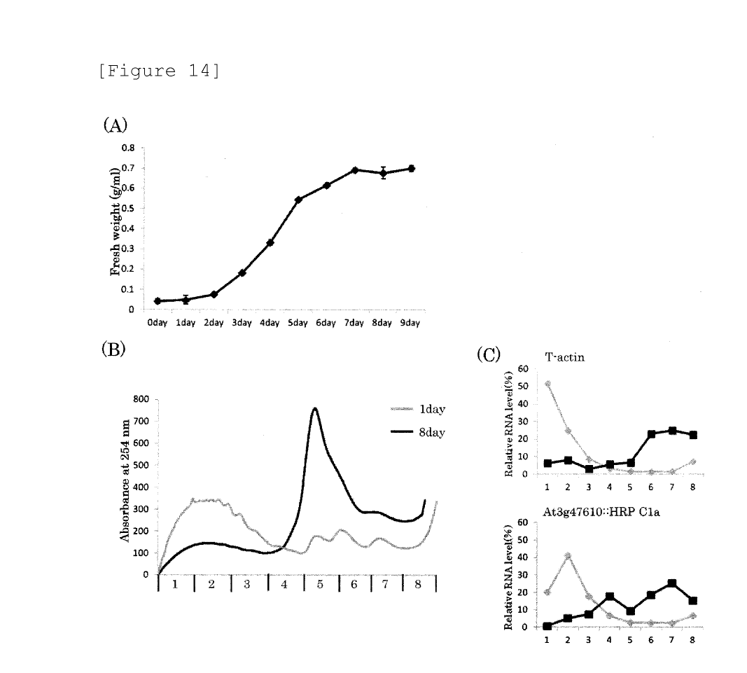

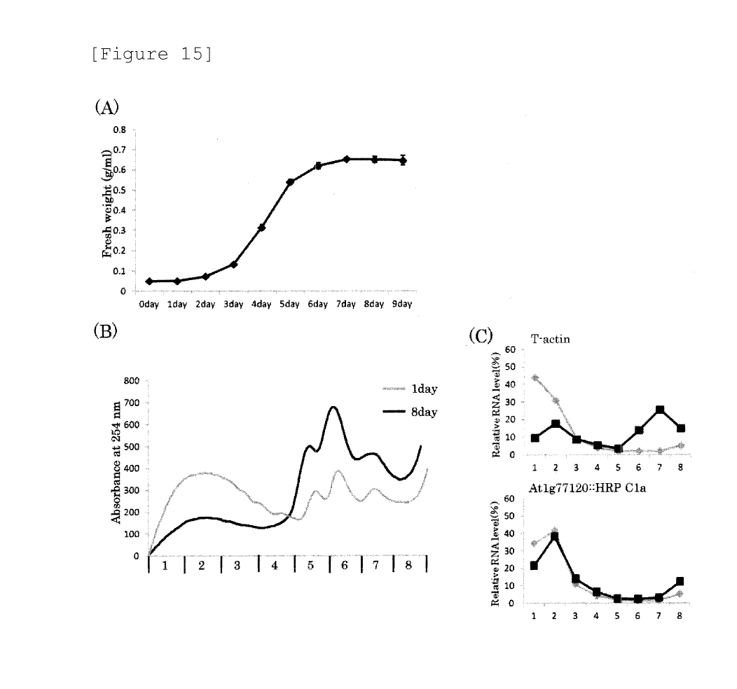

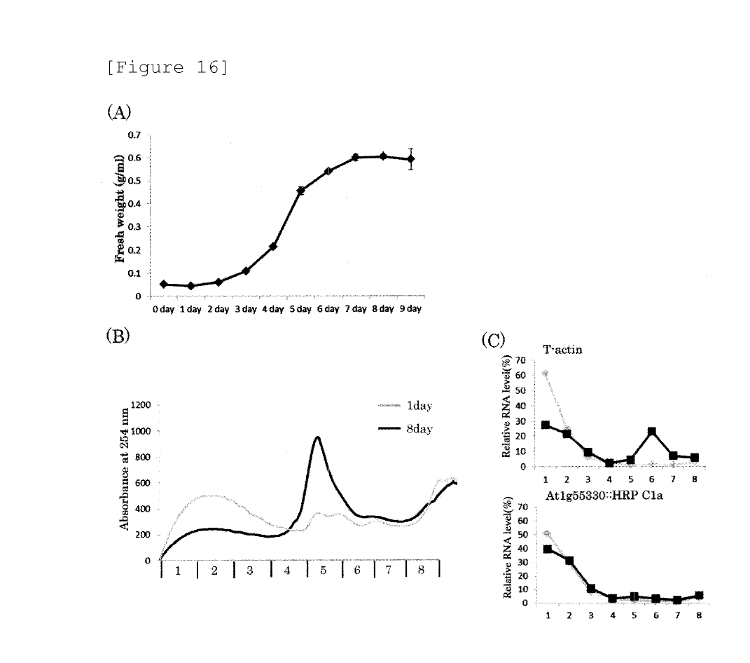

The fresh weights of the cells were measured at the time of culturing to create growth curve (FIG. 14A). The cells on day 1 of culture (first culture period) and the cells on day 8 of culture (late culture period) were collected to perform the same analysis as above. The results of day 8 of culture showed a decrease in polysome fractions and an increase in non-polysome fractions (FIG. 14B). The behavior of the mRNAs were individually analyzed by quantitative RT-PCR to thereby confirm that translation of the tobacco actin (T-actin) mRNA was repressed on day 8 of culture, as is the case with act2 of Arabidopsis thaliana. In HPP C1a containing the 5′ UTR of gene At3g47610 whose translation was repressed in the late period of culturing Arabidopsis thaliana (FIG. 5B), translational repression occurred on day 8 of culture of the tobacco cultured cells (FIG. 14C), as is the case with Arabidopsis thaliana.

Transformed Tobacco Cultured Cells (At1g77120 5′ UTR::HRP C1a)

The fresh weights of the cells were measured at the time of culturing to create growth curve (FIG. 15A). The cells on day 1 of culture (first culture period) and the cells on day 8 of culture (late culture period) were collected to perform the same analysis as above. The results of day 8 of culture showed a decrease in polysome fractions and an increase in non-polysome fractions (FIG. 15B). The behavior of the mRNAs was individually analyzed by quantitative RT-PCR to thereby confirm that the tobacco actin (T-actin) mRNA shifted to non-polysome fractions on day 8 of culture. In contrast, regarding the HRP C1a mRNA containing the 5′ UTR of At1g77120 whose translation was not repressed in the late period of culturing Arabidopsis thaliana, the majority remained in the polysome fractions on day 8 of culture, indicating that the translation was not repressed (FIG. 15C).

Transformed Tobacco Cultured Cells (At1g55330 5′ UTR::HRP C1a)

The fresh weights of the cells were measured at the time of culturing to create growth curve (FIG. 16A). The cells on day 1 of culture (first culture period) and the cells on day 8 of culture (late culture period) were collected to perform the same analysis as above. The results of day 8 of culture showed a decrease in polysome fractions and an increase in non-polysome fractions (FIG. 16B). The behavior of the mRNAs was individually analyzed by quantitative RT-PCR to thereby confirm that the tobacco actin (T-actin) mRNA shifted to non-polysome fractions on day 8 of culture. In contrast, regarding the HRP C1a mRNA containing the 5′ UTR of At1g55330 whose translation was not repressed in the late period of culturing Arabidopsis thaliana, the majority remained in the polysome fractions on day 8 of culture, indicating that the translation was not repressed (FIG. 16C). These results are similar to the results obtained in relation to Arabidopsis thaliana, and it is thereby confirmed that the 5′ UTRs that are isolated from Arabidopsis thaliana and that have an ability to maintain translation under stress due to absence of conditions required for the growth can also exert the effects in other plants (e.g., tobacco).

1. A method for escaping translational repression of a protein encoded by mRNA, the repression being induced by stress due to absence of conditions required for the growth,
the method comprising the step of culturing a plant cell transformed with a recombinant DNA molecule encoding mRNA containing 5′ UTR defined in (a) or (b) below, the culturing being carried out under stress due to absence of conditions required for the growth:
(a) 5′ UTR having a base sequence of SEQ ID NO: 1, 2, 3, 4, 5, or 6; or
(b) 5′ UTR having the base sequence of the 5′ UTR of (a) in which one or more bases are replaced, deleted, or added, and which escapes translational repression induced by stress due to absence of conditions required for the growth.

2. The method according to claim 1, wherein the stress due to absence of conditions required for the growth is at least one stress selected from the group consisting of nutrient-starvation stress and hypoxic stress.

3. The method according to claim 1, wherein the plant cell is an Arabidopsis thaliana cultured cell or a tobacco cultured cell.

4. A method for producing a protein by escaping translational repression of a protein encoded by mRNA, the repression being induced by stress due to absence of conditions required for the growth,
the method comprising the step of culturing a plant cell transformed with a recombinant DNA molecule encoding mRNA containing 5′ UTR defined in (a) or (b) below, the culturing being carried out under stress due to absence of conditions required for the growth:
(a) 5′ UTR having a base sequence of SEQ ID NO: 1, 2, 3, 4, 5, or 6; or
(b) 5′ UTR having the base sequence of the 5′ UTR of (a) in which one or more bases are replaced, deleted, or added, and which escapes translational repression induced by stress due to absence of conditions required for the growth.

5. A recombinant DNA containing a region encoding the RNA having a base sequence of SEQ ID NO: 2, 3, 5, or 6.

6. The method according to claim 2, wherein the plant cell is an Arabidopsis thaliana cultured cell or a tobacco cultured cell.
